(19) United States
(12) Patent Application Publication
(10) Pub. No.: US 2024/0199560 A1
(43) Pub. Date: Jun. 20, 2024

(54) PROCESS FOR PREPARING (2Z)-2-(PHENYLIMINO)-1,3-THIAZOLIDINE-4-ONE-SULFOXIDE DERIVATIVES IN AN ENANTIOMERICALLY ENRICHED FORM

(71) Applicant: Bayer Aktiengesellschaft, Leverkusen (DE)

(72) Inventors: Thomas HIMMLER, Odenthal (DE); Julia Johanna HAHN, Duesseldorf (DE); Daniel GALLENKAMP, Wuppertal (DE)

(21) Appl. No.: 18/551,614

(22) PCT Filed: Mar. 22, 2022
(86) PCT No.: PCT/EP2022/057479

§ 371 Date: Sep. 21, 2023

(30) Foreign Application Priority Data
Mar. 26, 2021 (EP) 21165202.9

(51) Int. Cl. C07D 277/54; B01J 31/04; B01J 31/22

(52) U.S. Cl. CPC C07D 277/54; B01J 31/04; B01J 31/2217; B01J 2231/70; B01J 2531/46; B01J 2531/842

(57) ABSTRACT

The present invention relates to a catalytic process for preparing 2-(phenylimino)-1,3-thiazolidin-4-one sulfoxide derivatives of formula (I) in enantiomerically pure or enantiomerically enriched form,

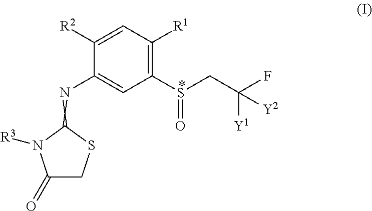

in which Y¹, Y², R¹, R² and R³ are as defined in the description.

The present invention relates to a catalytic process for preparing 2-(phenylimino)-1,3-thiazolidin-4-one sulfoxide derivatives in enantiomerically pure or in an enantiomerically enriched form.

The chemical synthesis of 2-(phenylimino)-1,3-thiazolidin-4-one sulfoxide derivatives is known in principle and described for example in international patent application (WO 2013/092350).

Chiral sulfoxides and corresponding derivatives are of great importance in the pharmaceutical and agrochemical industries. The preparation of enantiomerically pure chiral sulfoxides not only avoids waste in the production process but also avoids potentially harmful side effects that may result from the undesired enantiomer (Nugent et al., Science 1993, 259, 479; Noyori et al., CHEMTECH 1992, 22, 360).

The enantioselective synthesis of chiral sulfoxides is described in the literature. Review articles which describe this methodology may be found for example in H. B. Kagan and I. Ojima (ed.) “Catalytic Asymmetric Synthesis (2^{nd} Edition)” Wiley-VCH: New York 2000, 327-356; M. Beller and C. Bolm (ed.) “Transition Metals for Organic Synthesis: Building Blocks and Fine Chemicals, Second Revised and Enlarged Ed.” Wiley-VCH 2004, 479-495; E. Wojaczyńska and J. Wojaczyński in Chem. Rev. 2010, 110, 4303-4356; G. E. O′Mahony in ARKIVOC 2011, 1-110. In addition to the classically metal-catalysed methods for synthesis of enantiomerically enriched chiral sulfoxides the literature also describes enzymatic processes (K. Faber in “Biotransformations in Organic Synthesis (6th Edition)”, Springer: Berlin Heidelberg 2011; H. L. Holland, Nat. Prod. Rep., 2001, 18, 171-181). The enzymatic methods are predominantly substrate-specific and their industrial implementation is also very costly and complex. For example monooxygenases and peroxidases are important enzyme classes which are capable of catalysing for a multiplicity of sulfides the oxidation thereof to the corresponding sulfoxides (S. Colonna et al., Tetrahedron Asymmetry 1993, 4, 1981). However it has been shown that the stereochemical result of enzymatic oxidation depends strongly on the sulfide structure.

An often employed process for enantioselective oxidation of thioethers is Kagan's modified method of the known Sharpless epoxidation with chiral titanium complexes (J. Am. Chem. Soc. 1984, 106, 8188-8193). The chiral titanium complex consisting of Ti(OⁱPr)₄ and (+)- or (−)-diethyl tartrate (DET) is “deactivated” with one equivalent of water and catalyses the enantioselective sulfide oxidation of aryl alkyl sulfides. However Kagan's reagent only achieved good results with high proportions of DET (for example a mixing ratio of Ti(OⁱPr)₄/DET/H₂O=1:2:1) and an organic peroxide (for example tert-butylhydroperoxide). The good enantioselectivity of the described titanium complexes is accompanied by a low catalytic activity which explains the necessary molar ratio between substrate and catalyst. This process makes it possible to achieve direct oxidation of simple aryl alkyl sulfides, for example aryl methyl sulfides, to afford optically active sulfoxides. It was found that asymmetric oxidation of functionalized alkyl sulfides for example proceeds with moderate enantioselectivity under these conditions.

While Pasini et al. were able to oxidize phenyl methyl sulfide with small amounts of chiral oxotitanium(IV) complexes and hydrogen peroxide this was done with poor enantiomeric excesses of ee<20% (Gaz. Chim. Ital. 1986, 116, 35-40). Furthermore, titanium-catalysed processes entail very costly and complex workups and this is very disadvantageous for an economic process on an industrial scale.

A further method is based on vanadium(IV)/iron(III) complexes as efficient catalysts for sulfide oxidation. The chiral catalysts are prepared in situ from VO(acac)₂ (Synlett 1998, 12, 1327-1328; Euro. J. Chem. 2009, 2607-2610) or Fe(acac)₃ (Angew. Chem. Int. Ed. 2003, 42, 5487-5489; Angew. Chem. Int. Ed. 2004, 43, 4225-4228) as precursors together with a Schiff base. However, this method is limited to simple and non-fluorinated aryl alkyl thioethers such as p-tolyl methyl sulfide for example.

For sulfide oxidation of iron(III) complexes using hydrogen peroxide it is further described that the use of enantiomerically pure chiral Schiff base ligands results in enantiomerically enriched chiral sulfoxides (Chem. Eur. J. 2005, 11, 1086-1092). The substituents in the ligand are of very great importance for chiral induction but these effects cannot be rationally explained, let alone predicted.

It has likewise already become known that in the sulfide oxidation of iron(III) complexes using hydrogen peroxide both chemical conversion and chiral induction may be increased using additives (Chem. Eur. J. 2005, 11, 1086-1092). Additives described include carboxylic acids and in particular their corresponding alkali metal and ammonium salts. Especially benzoic acids with electron donating radicals in the para position, for example p-methoxy- or p-dimethylaminobenzoic acid, and sterically hindered benzoic acids such as 2,4,6-trimethylbenzoic acid can result in improved yields and higher enantiomeric excesses in the oxidation of thioanisoles. However, precisely predicting these effects is not possible.

The enantiomers of (2Z)-2-(phenylimino)-1,3-thiazolidin-4-one sulfoxide derivatives obtained as a racemic mixture by literature methods have hitherto been obtained via a costly and complex separation using HPLC on chiral phases. However, chromatographic separation of enantiomers on chiral stationary phases is generally unsuitable for comparatively large amounts of active ingredient, but serves merely for provision of relatively small amounts. The utilization of HPLC on chiral phases is moreover extremely costly because of the high cost of these materials and the considerable time investment required, especially on the preparative scale. A catalytic process for enantioselective preparation of 2-(phenylimino)-1,3-thiazolidin-4-one sulfoxide derivatives which is also performable efficiently from an industrial and economic standpoint is not derivable from the prior art.

International patent application WO 2011/006646 does provide a catalytic process which especially allows preparation of enantiomerically enriched 3-(1H-1,2,4-triazolyl)sulfoxide derivatives using an iron(III) catalyst. However, the document discloses methylene chloride as a particularly suitable solvent for these reactions, this solvent being less well-suited to use on an industrial scale. It lacks a specific indication that the disclosed process can also be successfully utilized for the preparation of enantiomerically enriched 2-(phenylimino)-1,3-thiazolidin-4-one sulfoxide derivatives as well as an indication of additives and/or solvents suitable therefor.

International patent application WO 2013/092350 provides a catalytic process which in particular allows preparation of enantiomerically enriched N-arylamidine-substituted trifluoroethylsulfoxide derivatives. A vanadium(IV)-based catalyst system in chloroform is described as particularly suitable. However, this document mentions neither the use of Fe(III) or other transition metal-based systems nor does it mention alternative solvents or mixtures of solvents which could substitute the chloroform that is rather unsuitable for industrial scale usage.

Having regard to the prior art identified there was therefore a continuing need for a simplified industrially and economically performable catalytic process for enantioselective preparation of 2-(phenylimino)-1,3-thiazolidin-4-one sulfoxide derivatives, in particular of substituted, fluorinated 2-(phenylimino)-1,3-thiazolidin-4-one sulfoxide derivatives. The 2-(phenylimino)-1,3-thiazolidin-4-one sulfoxide derivatives obtainable by this sought process should preferably be obtained with high yield, high chemical purity and high optical purity, i.e. high enantiomeric excess, preferably expressed as the ee value. In particular, the process sought should enable the desired target compounds to be obtained without the need for complex purification methods such as chiral chromatography. The process sought should moreover preferably permit the use of solvents suitable for the industrial scale.

It bas surprisingly been found that 2-(phenylimino)-1,3-thiazolidin-4-one sulfoxide derivatives may be prepared in enantiomerically enriched form in a transition metal-catalysed, in particular Fe(III)-catalysed, process using suitable additives. This is all the more surprising since no Fe(III)-catalysed processes with 2-(phenylimino)-1,3-thiazolidin-4-one sulfoxide derivatives have hitherto been reported and those skilled in the art would have expected the present thiazolidinone group of these compounds to interact counterproductively with an Fe(III) ligand complex, thus precisely not achieving satisfactory yield and/or optical purity. It was moreover not foreseeable that the (R) enantiomer of the ligand is required for the stereoselective synthesis of the desired (R) sulfoxide and not, as described in WO2011/006646, the (S) enantiomer of the ligand.

The present invention accordingly provides a process for preparing 2-(phenylimino)-1,3-thiazolidin-4-one sulfoxide derivatives of formula (I) in enantiomerically pure or enantiomerically enriched form

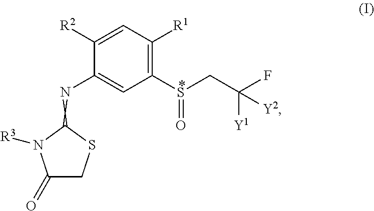

in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro, and
R³ is hydrogen or optionally substituted C₆-C₁₀-aryl, (C₁-C₁₂)alkyl or (C₁-C₁₂)haloalkyl, wherein the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, in particular from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy,
characterized in that it comprises reacting a sulfide of formula (II):

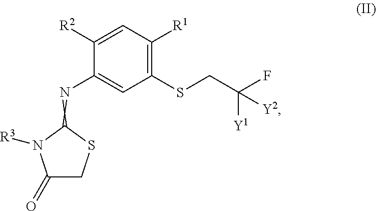

in which Y¹, Y², R¹, R² and R³ are as defined above, in the presence of an enantiomerically enriched chiral catalyst, an additive which is the salt of an organic acid and an oxidizing agent.

The compounds of formulae (I) and (II) may be present as the E- or Z-isomer or as a mixture of these isomers. This is indicated by the crossed double bond in the formulae (I) and (II). In an individual embodiment of the invention, the compound is in each case in the form of the E-isomer. In another individual embodiment of the invention, the compound is in each case in the form of the Z-isomer. In another individual embodiment of the invention, the compound is in the form of a mixture of the E- and Z-isomers.

Preferred, particularly preferred and very particularly preferred definitions of the radicals Y¹, Y², R¹, R² and R³ appearing in formulae (I) and (II) mentioned hereinabove are elucidated below.

It is preferable when
Y¹ and Y² are each independently fluorine, chlorine or hydrogen, and
R¹ and R² are each independently fluorine, chlorine, (C₁-C₃)alkyl or hydrogen, and
R³ is hydrogen or optionally substituted phenyl, (C₁-C₆)alkyl or (C₁-C₆)haloalkyl, wherein the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, especially from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy.

It is particularly preferable when
Y¹ and Y² are each independently fluorine or hydrogen, and
R¹ and R² are each independently fluorine, chlorine, hydrogen or methyl, and
R³ is hydrogen, (C₁-C₆)alkyl or (C₁-C₆)haloalkyl.

It is very particularly preferable when
Y¹ and Y² are fluorine, and
R¹ and R² are each independently fluorine or methyl and
R³ is (C₁-C₆)haloalkyl.

It is exceptionally preferable when
Y¹ and Y² are fluorine,
R¹ is methyl,
R² is fluorine and
R³ is CH₂CF₃.

It has been found that, surprisingly, the process according to the invention makes it possible to prepare the chiral (2-(phenylimino)-1,3-thiazolidin-4-one sulfoxide derivatives of formula (I) in good yields and in high chemical and optical purity and consequently with high enantiomeric excesses (preferably expressed as the ee value). The process according to the invention moreover makes it possible to use solvents suitable for the industrial scale. A further advantage is that the process according to the invention makes it possible to obtain the desired target compounds without the need for complex purification methods such as chiral chromatography.

Depending on the preparation conditions the process according to the invention affords compounds of formula (I) in an enantiomeric ratio of 50.5:49.5 to 100:0 (R):(S) enantiomer or (S):(R) enantiomer. The (R) enantiomer of the compound of formula (I) is preferred according to the invention.

Enantiomeric purity may if necessary be increased by various methods. Such methods are known to those skilled in the art and especially include the preferred crystallization from an organic solvent or a mixture of organic solvent with water or a mixture of organic solvents.

The process according to the invention can be illustrated by the following scheme (I):

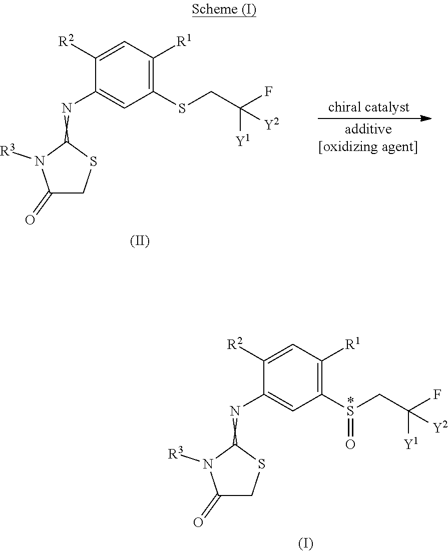

wherein Y¹, Y², R¹, R² and R³ are as defined hereinabove.

### GENERAL DEFINITIONS

In the context of the present invention, the term “halogens” (Hal) encompasses, unless otherwise defined, elements selected from the group consisting of fluorine, chlorine, bromine and iodine, preference being given to using fluorine, chlorine and bromine, and particular preference to using fluorine and chlorine.

Optionally substituted groups may be singly or multiply substituted; if multiply substituted, the substituents may be identical or different. Unless otherwise stated at the relevant position, the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, in particular from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy.

Alkyl groups substituted by one or more halogen atoms (Hal) are, for example, selected from trifluoromethyl (CF₃), difluoromethyl (CHF₂), CF₃CH₂, ClCH₂ or CF₃CCl₂.

Alkyl groups in the context of the present invention are, unless otherwise defined, linear, branched or cyclic saturated hydrocarbon groups.

The definition C₁-C₁₂-alkyl encompasses the widest range defined herein for an alkyl group. Specifically, this definition encompasses, for example, methyl, ethyl, n-propyl, isopropyl, n-butyl, isobutyl, sec-butyl and t-butyl, n-pentyl, n-hexyl, 1,3-dimethylbutyl, 3,3-dimethylbutyl, n-heptyl, n-nonyl, n-decyl, n-undecyl, n-dodecyl.

Aryl groups in the context of the present invention are, unless otherwise defined, aromatic hydrocarbon groups, which may comprise one, two or more heteroatoms (selected from O, N, P and S).

Specifically, this definition encompasses, for example, cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Alkylaryl groups in the context of the present invention, unless defined differently, are aryl groups substituted by alkyl groups, which have one alkylene chain and may have, in the aryl skeleton, one or more heteroatoms (selected from O, N, P and S).

The term enantiomerically enriched is to be understood as meaning the presence of an enantiomeric mixture of such a compound in which a certain enantiomer of this compound is present in a relatively large amount compared to the other enantiomer of this compound. In the case of two possible enantiomers of a compound the enantiomeric mixture accordingly contains more than 50% of one enantiomer. The proportion of an enantiomer in an enantiomerically enriched mixture is preferably more than 50% and increasingly preferably more than 60%, 65%, 70%, 75, 80%, 85%, 90%, 92.5%, 95%, 96%, 97%, 98%, 98.5%, 99%, 99.1%, 99.2%, 99.3%, 99.4%, 99.5%, 99.6%, 99.7% and 99.75%, in each case based on the total amount of both enantiomers of the compound. In this regard in the context of the present patent application an enantiomeric mixture is also referred to as enantiomerically pure above the presence of more than 99% of one enantiomer in said enantiomeric mixture.

Thus, the enantiomeric excess may be between 0% ee and 100% ee. The enantiomeric excess is an indirect measure of the enantiomeric purity of a compound and indicates the proportion of a pure enantiomer in a mixture, the remaining portion of which is the racemate of the compound.

Suitable methods for determining the enantiomeric excess are familiar to those skilled in the art. Examples include HPLC on chiral stationary phases and NMR analyses with chiral shift reagents.

The chiral catalyst of the process according to the invention is a chiral metal-ligand complex. This chiral metal-ligand complex is prepared from a chiral ligand and a transition metal or preferably a transition metal derivative. The transition metal derivative is preferably selected from molybdenum, zirconium, iron, manganese and titanium derivatives and particularly preferably iron derivatives. These derivatives are very particularly preferably employed in the form of the transition metal(II) or (III) halides, transition metal(II) or (III) carboxylates or transition metal(II) or (III) acetylacetonates.

The transition metal derivative is more preferably selected from an iron or titanium derivative, in particular titanium and iron halides, carboxylates and acetylacetonates, wherein iron(II) and iron(III) acetylacetonate are very particularly preferred.

The chiral ligand is a compound capable of forming a chiral metal-ligand complex with the transition metal derivative. Such ligands are preferably selected from compounds having at least two heteroatoms suitable for complexing to the metal (for example O, N, P, S). Preferred chiral ligands are those of formula (III):

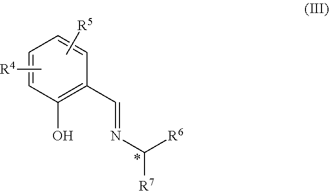

wherein, in formula (III),
R⁴ and R⁵ are each independently hydrogen, (C₁-C₆)alkyl, (C₁-C₆)haloalkyl, (C₁-C₆)alkylphenyl, phenyl, halogen, cyano, nitro, cyano(C₁-C₆)alkyl, hydroxy(C₁-C₆)alkyl, (C₁-C₆)alkoxycarbonyl(C₁-C₆)alkyl, (C₁-C₆)alkoxy, (C₁-C₆)haloalkoxy or (C₁-C₆)alkoxy(C₁-C₆)alkyl,
R⁶ is (C₁-C₆)alkyl, halogen-, cyano-, nitro-, amino-, hydroxy- or phenyl-substituted (C₁-C₆)alkyl, carboxyl, carbonyl(C₁-C₆)alkyl, (C₁-C₆)alkoxycarbonyl(C₁-C₆)alkyl, (C₁-C₆)alkoxy(C₁-C₆)alkyl, (C₁-C₆)alkoxy or di(C₁-C₆)alkoxy(C₁-C₆)alkyl,
R⁷ is hydrogen, (C₁-C₆)alkyl, (C₁-C₆)alkylphenyl, aryl or aryl(C₁-C₆)alkyl,
and chiral carbon atoms are identified by *.

It is preferable when
R⁴ and R⁵ are each independently hydrogen, (C₁-C₄)alkyl, (C₁-C₄)alkylphenyl, phenyl, halogen, cyano, nitro, cyano(C₁-C₄)alkyl, hydroxy(C₁-C₄)alkyl, (C₁-C₄)alkoxycarbonyl(C₁-C₄)alkyl or (C₁-C₄)alkoxy(C₁-C₄)alkyl,
R⁶ is (C₁-C₃)alkyl, halogen-, cyano-, nitro-, amino-, hydroxy- or phenyl-substituted (C₁-C₃)alkyl, carboxyl, carbonyl(C₁-C₃)alkyl, (C₁-C₃)alkoxycarbonyl(C₁-C₃)alkyl, (C₁-C₃)alkoxy(C₁-C₃)alkyl, (C₁-C₃)alkoxy or di(C₁-C₃)alkoxy(C₁-C₃)alkyl, and
R⁷ is hydrogen, (C₁-C₄)alkyl, (C₁-C₄)alkylphenyl, aryl or aryl(C₁-C₄)alkyl.

It is particularly preferable when
R⁴ and R⁵ are each independently hydrogen, (C₁-C₄)alkyl, phenyl, halogen, cyano, nitro, hydroxy(C₁-C₄)alkyl, (C₁-C₄)alkoxycarbonyl(C₁-C₄)alkyl or (C₁-C₄)alkoxy(C₁-C₄)alkyl,
R⁶ is halogen-, cyano-, nitro-, amino-, hydroxy- or phenyl-substituted (C₁-C₃)alkyl or carboxyl and
R⁷ is tert-butyl, isopropyl, benzyl or phenyl.

It is very particularly preferable when
R⁴ and R⁵ are independently hydrogen, chlorine, bromine, iodine or tert-butyl,
R⁶ is hydroxy-substituted C₁-alkyl and
R⁷ is tert-butyl or isopropyl.

It is exceptionally preferable when
R⁴ and R⁵ are each independently hydrogen or chlorine
R⁶ is hydroxy-substituted C₁-alkyl and
R⁷ is tert-butyl.

The chiral ligands of formula (III) are employed as enantiomerically enriched compounds. It is preferable when the optical purity of the ligands expressed as the ee value (enantiomeric excess=(enantiomer present in excess minus enantiomer present in deficiency) divided by (enantiomer present in excess plus enantiomer present in deficiency) multiplied by 100) is between ee=40% and ee=100%, particularly preferably between ee=80% and ee=100%.

More preferred chiral ligands are those of formula (IIIa):

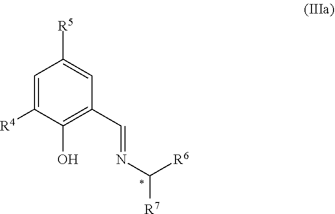

wherein in formula (IIIa)
R⁴ and R⁵ are each independently hydrogen, (C₁-C₆)alkyl, (C₁-C₆)alkylphenyl, phenyl, halogen, cyano, nitro, cyano(C₁-C₆)alkyl, hydroxy(C₁-C₆)alkyl, (C₁-C₆)alkoxycarbonyl(C₁-C₆)alkyl or (C₁-C₆)alkoxy(C₁-C₆)alkyl,
R⁶ is (C₁-C₆)alkyl, halogen-, cyano-, nitro-, amino-, hydroxy- or phenyl-substituted (C₁-C₆)alkyl, carboxyl, carbonyl(C₁-C₆)alkyl, (C₁-C₆)alkoxycarbonyl(C₁-C₆)alkyl, (C₁-C₆)alkoxy(C₁-C₆)alkyl, (C₁-C₆)alkoxy or di(C₁-C₆)alkoxy(C₁-C₆)alkyl,
R⁷ is hydrogen, (C₁-C₆)alkyl, (C₁-C₆)alkylphenyl, aryl or aryl(C₁-C₆)alkyl,
and chiral carbon atoms are identified by *.

It is preferable when
R⁴ and R⁵ are each independently hydrogen, (C₁-C₄)alkyl, (C₁-C₄)alkylphenyl, phenyl, halogen, cyano, nitro, cyano(C₁-C₄)alkyl, hydroxy(C₁-C₄)alkyl, (C₁-C₄)alkoxycarbonyl(C₁-C₄)alkyl or (C₁-C₄)alkoxy(C₁-C₄)alkyl,
R⁶ is (C₁-C₃)alkyl, halogen-, cyano-, nitro-, amino-, hydroxy- or phenyl-substituted (C₁-C₃)alkyl, carboxyl, carbonyl(C₁-C₃)alkyl, (C₁-C₃)alkoxycarbonyl(C₁-C₃)alkyl, (C₁-C₃)alkoxy(C₁-C₃)alkyl, (C₁-C₃)alkoxy or di(C₁-C₃)alkoxy(C₁-C₃)alkyl, and
R⁷ is hydrogen, (C₁-C₄)alkyl, (C₁-C₄)alkylphenyl, aryl or aryl(C₁-C₄)alkyl.

It is particularly preferable when
R⁴ and R⁵ are each independently hydrogen, (C₁-C₄)alkyl, phenyl, halogen, cyano, nitro, hydroxy(C₁-C₄)alkyl, (C₁-C₄)alkoxycarbonyl(C₁-C₄)alkyl or (C₁-C₄)alkoxy(C₁-C₄)alkyl,
R⁶ is halogen-, cyano-, nitro-, amino-, hydroxy- or phenyl-substituted (C₁-C₃)alkyl or carboxyl and
R⁷ is tert-butyl, isopropyl, benzyl or phenyl.

It is very particularly preferable when
R⁴ and R⁵ are each independently hydrogen, chlorine, bromine, iodine or tert-butyl,
R⁶ is hydroxy-substituted C₁-alkyl and
R⁷ is tert-butyl or isopropyl.

It is exceptionally preferable when
R⁴ and R⁵ are each independently hydrogen or chlorine
R⁶ is hydroxy-substituted C₁-alkyl and
R⁷ is tert-butyl.

The chiral ligands of formula (IIIa) are employed as enantiomerically enriched compounds. It is preferable when the optical purity of the ligands expressed as the ee value (enantiomeric excess=(enantiomer present in excess minus enantiomer present in deficiency) divided by (enantiomer present in excess plus enantiomer present in deficiency) multiplied by 100) is between ee=40% and ee=100%, particularly preferably between ee=80% and ee=100%.

In a separate embodiment of the invention, the chiral ligand of formula (III) or of formula (IIIa) is employed in the (R) configuration in order to obtain the R enantiomer of the compound of formula (I) in enriched form.

In a further separate embodiment of the invention, the chiral ligand of formula (III) or of formula (IIIa) is employed in the (S) configuration in order to obtain the S enantiomer of the compound of formula (I) in enriched form.

In a further separate embodiment of the invention, the chiral ligand of formula (III) or of formula (IIIa) is employed in the (R) configuration in order to obtain the S enantiomer of the compound of formula (I) in enriched form.

In a further separate embodiment of the invention, the chiral ligand of formula (III) or of formula (IIIa) is employed in the (S) configuration in order to obtain the R enantiomer of the compound of formula (I) in enriched form.

The chiral metal-ligand complex is obtained by reaction of a transition metal derivative and a chiral ligand separately or in the presence of the sulfide. The ratio of transition metal derivative to chiral ligand is in the range from 10:1 to 1:10, preferably in the range from 1:1 to 1:10, particularly preferably in the range from 1:1 to 1:5 and very particularly preferably in the range 1:1 to 1:3. The ligands may be prepared by known methods (for example Adv. Synth. Catal. 2005, 347, 1933-1936).

The usage of chiral metal ligand complex based on the sulfide of formula (II) is preferably in the range from 0.01 to 20 mol %, preferably from 0.1 to 10 mol %, particularly preferably from 0.5 to 7 mol % and very particularly preferably from 0.5 to 5 mol %. A higher usage of chiral metal-ligand complex is possible but generally not economically sensible. The chiral metal-ligand complex/the constituents thereof may either already be present at commencement of the reaction or else may in part be added during the reaction until attainment of the intended total amount.

The additive is the salt of an organic acid. The salt is especially an alkali metal or ammonium salt, wherein lithium, sodium or potassium salts are in turn preferred among these.

Preferred additives are those of formula (IV):

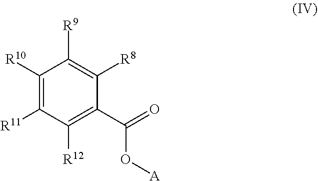

wherein in formula (IV)
R⁸, R⁹, R¹⁰, R¹¹ and R¹² are each independently hydrogen, (C₁-C₆)alkyl, (C₁-C₆)haloalkyl, (C₁-C₆)haloalkoxy, (C₁-C₆)alkylphenyl, phenyl, halogen, cyano, nitro, (C₁-C₆)alkoxy, cyano(C₁-C₆)alkyl, hydroxy(C₁-C₆)alkyl, (C₁-C₆)alkoxycarbonyl(C₁-C₆)alkyl, (C₁-C₆)alkoxy(C₁-C₆)alkyl or aminodi(C₁-C₆)alkyl and
A is lithium, sodium, potassium or an NR¹³R¹⁴R¹⁵R¹⁶ radical,
wherein
R¹³, R¹⁴, R¹⁵ and R¹⁶ are each independently hydrogen, benzyl or (C₁-C₆)alkyl.

It is preferable when
R⁸, R⁹, R¹¹ and R¹² are each independently hydrogen, (C₁-C₄)alkyl or (C₁-C₄)alkoxy,
R¹⁰ is hydrogen, (C₁-C₄)alkyl, (C₁-C₄)alkoxy or aminodi(C₁-C₄)alkyl and
A is lithium, sodium, potassium or ammonium.

It is particularly preferable when
R⁸, R⁹, R¹¹ and R¹² are each independently hydrogen or methoxy,
R¹⁰ is hydrogen, methoxy or dimethylamino and
A is lithium, sodium, potassium or ammonium.

It is very particularly preferable when
R⁸, R⁹, R¹¹ and R¹² are hydrogen,
R¹⁰ is hydrogen or methoxy or dimethylamino and
A is lithium, sodium or potassium.

The usage of additive based on the sulfide of formula (II) is preferably in the range from 0.1 to 20 mol %, particularly preferably from 0.5 to 10 mol % and very particularly preferably from 1 to 8 mol %. A higher usage of additive is possible but generally not economically sensible.

The preferred, particularly preferred and very particularly preferred additives (IV) where A=lithium, sodium, potassium or ammonium may either be prepared separately and supplied to the reaction mixture as these salts or the additive (IV) where A=hydrogen is employed and the salt is prepared in situ by addition of a suitable amount of a lithium base, sodium base, potassium base or ammonia. Particularly preferred in this regard are lithium hydroxide, sodium hydroxide, potassium hydroxide or ammonia.

The reaction of the sulfide of formula (II) to afford the compound of formula (I) is preferably carried out in the presence of a solvent. Suitable solvents especially include: tetrahydrofuran (THF), dioxane, diethyl ether, diglyme, methyl tert-butyl ether (MTBE), tert-amyl methyl ether (TAME), dimethyl ether (DME), 2-methyl-THF, acetonitrile (ACN), acetone, butyronitrile, toluene, anisole, o-xylene, m-xylene, p-xylene, ethylbenzene, mesitylene, ethyl acetate, isopropyl acetate, butyl acetate, pentyl acetate, methyl isobutyl ketone, alcohols such as methanol, ethanol, propanol, butanol, ethylene glycol, ethylene carbonate, propylene carbonate, N,N-dimethylacetamide (DMAc), N,N-dimethylformamide (DMF), N-methylpyrrolidone, hydrohalocarbons and aromatic hydrocarbons, especially hydrochlorocarbons, such as tetrachloroethylene, tetrachloroethane, dichloropropane, methylene chloride (dichloromethane, DCM), dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, in particular 1,2-dichlorobenzene, chlorotoluene, trichlorobenzene; 4-methoxybenzene, fluorinated aliphatics and aromatics such as trichlorotrifluoroethane, benzotrifluoride, 4-chlorobenzotrifluoride and water. It is also possible to use solvent mixtures.

Preferred solvents are methylene chloride, chloroform, 1,2-dichloroethane, chlorobenzene, 1,2-dichlorobenzene, acetonitrile, acetone, toluene, anisole, o-xylene, m-xylene, p-xylene, ethylbenzene, ethyl acetate, methyl tert-butyl ether (MTBE), tetrahydrofuran (THF), N,N-dimethylacetamide (DMAc), N,N-dimethylformamide (DMF), ethanol or mixtures thereof.

Particularly preferred solvents are methylene chloride, 1,2-dichloroethane, chlorobenzene, anisole, toluene, o-xylene, m-xylene, p-xylene, ethylbenzene or mixtures thereof.

Very particularly preferred solvents are toluene, o-xylene, m-xylene, p-xylene, ethylbenzene, chlorobenzene, anisole and methylene chloride or mixtures thereof.

Exceptionally preferred solvents are toluene, o-xylene, m-xylene, p-xylene and methylene chloride or a mixture of o-xylene, m-xylene, p-xylene and ethylbenzene (technical-grade xylene).

The oxidizing agents that may be used for this reaction are not subject to any particular limitations. Suitable oxidizing agents for preparing the sulfoxides are for example inorganic peroxides, for example hydrogen peroxide, or organic peroxides such as for example alkyl hydroperoxides and arylalkyl hydroperoxides. A preferred oxidizing agent is hydrogen peroxide. The molar ratio of oxidizing agent to the sulfide of formula (II) is in the range from 0.9:1 to 5:1, preferably between 1.2:1 and 3.5:1.

The reaction is generally carried out at a temperature between −80° C. and 100° C., preferably between −10° C. and 60° C., very particularly preferably between −5° C. and 30° C.

The reaction is typically carried out at standard pressure, but may also be carried out at elevated or reduced pressure.

Products obtained after the process according to the invention have an enantiomeric ratio of 50.5:49.5 to 100:0, preferably of 75:25 to 100:0, particularly preferably of 90:10 to 100:0 (R):(S) enantiomer or (S):(R) enantiomer, very particularly preferably (R):(S) enantiomer. According to the invention preference is in each case given to the enantiomeric ratios exhibiting an excess of (R) enantiomer.

The desired compounds of formula (I) may be isolated for example by subsequent extraction and crystallization. If required, the enantiomeric excess may be increased significantly through subsequent crystallization. Such methods are known to those skilled in the art and especially include the preferred crystallization from an organic solvent or a mixture of organic solvent with water or a mixture of organic solvents. Preferred solvents for crystallization are 3-methyl-1-butanol and 1-butanol or their mixtures with methylcyclohexane.

The present invention is elucidated in detail by the examples that follow, although the examples should not be interpreted in such a manner that they restrict the invention.

### Preparation Examples

### Example 1: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

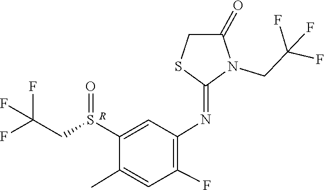

In a 5 l reaction vessel 1000 ml of toluene were initially charged and subsequently 16.1 g (0.046 mol) of iron(III) acetylacetonate, 43.1 g (0.091 mol) of 2-[(E)-{[(2R)-1-hydroxy-3,3-dimethylbutan-2-yl]imino}methyl]-4,6-diiodophenol and 13.3 g (0.09 mol) of sodium benzoate were added. A solution of 383.4 g (0.012 mol) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4one in 850 ml of toluene was subsequently added. 394 g (3.649 mol) of 31.5% hydrogen peroxide were then added over 90 minutes at an internal temperature of 22° C. to 27° C. The reaction mixture was then stirred at 25° C. overnight. The progress of the reaction was monitored by HPLC. The reaction mixture was diluted with 400 ml each of water and toluene at 5° C. to 10° C. and then stirred with 200 ml of a 39% aqueous sodium bisulfite solution. After phase separation, the aqueous phase was extracted with 400 ml of toluene. Concentrating the combined organic phases afforded 480.4 g of a dark oil. This was dissolved in 960 ml of methylene chloride and subjected to flash chromatography on 3.5 kg of silica gel (28 l methylene chloride, then 25 l methylene chloride (95%)+methyl tert-butyl ether (MTBE) (5%)). Removal of the solvent afforded 416.6 g of a tough orange resin. This resin was dissolved in 1200 ml of diisopropyl ether at 55° C. After distillative removal of 300 ml of diisopropyl ether the mixture was slowly cooled with stirring. The precipitated solid was filtered off, washed with 175 ml of diisopropyl ether and dried. This resulted in 352.7 g of yellowish solid having a purity of 99.2 HPLC fl %, corresponding to a yield of 87.9% of theory. Optical purity according to HPLC on a chiral phase is ee=94.6%.

1H-NMR (600 MHZ, CDCl₃): δ=2.4 (s, 3H), 3.4-3.5 (m, 1H), 3.97 (s, 2H), 4.5-4.6 (m, 1H), 7.1 (d, J=10.4 Hz, 1H), 7.6 (d, J=7.8 Hz, 1H) ppm.

### Example 2: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

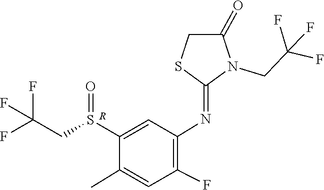

In the reaction vessel 0.75 ml of methylene chloride and 10.3 mg (0.029 mmol) of iron(III) acetylacetonate were initially charged. 17 mg (0.059 mmol) of 2,4-dichloro-6-[(E)-{[(2R)-1-hydroxy-3,3-dimethylbutan-2-yl]imino}methyl]phenol were subsequently added and the mixture was stirred for 5 minutes. 8.4 mg (0.059 mmol) of sodium benzoate, 246 mg (0.585 mmol) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one and a further 0.7 ml of methylene chloride were subsequently added. 165.9 mg (1.46 mmol) of 34% hydrogen peroxide were then slowly added at 20° C. to 22° C. Reaction monitoring by HPLC after 1 hour reaction time revealed at 100% conversion 93.6 fl % of the title compound with an ee of 98.9%.

### Examples 3 to 11

The synthesis described hereinabove in Example 2 was repeated with different ligands. The results are reported in Table 1 which follows.

TABLE 1
Oxidation of (2Z)-2-({2-fluoro-4-methyl-5-[(2,2,2-trifluoroethyl)sulfanyl)phenyl}imino)-
3-(2,2,2-trifluoroethyl)-1,3-thiazolidin-4-one according to Example 2 in the presence of different
ligands:

| | | | Conversion |
|---|---|---|---|
| Example | Ligand | ee [%] | [%] |
| 3 | 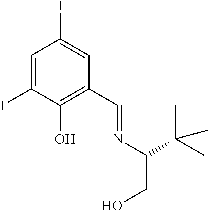 | 91.0 (R) enantiomer | 97 |
| 4 | 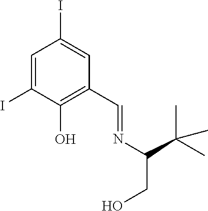 | 91.6 (S) enantiomer | 97 |
| 5 | 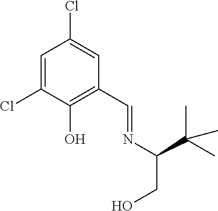 [SMILES: CC(C)(C)[C@@H](CO)/N=C/c1cc(Cl)cc(Cl)c1O] | 95.7 (S) enantiomer | 100 |
| 6 | 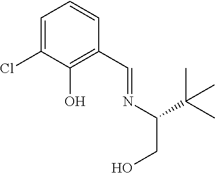 [SMILES: CC(C)(C)[C@H](CO)/N=C/c1cccc(Cl)c1O] | 97.1 (R) enantiomer | 100 |
| 7 | 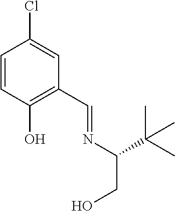 [SMILES: CC(C)(C)[C@H](CO)/N=C/c1cc(Cl)ccc1O] | 97.8 (R) enantiomer | 100 |
| 8 | 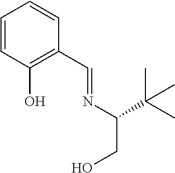 [SMILES: CC(C)(C)[C@H](CO)/N=C/c1ccccc1O] | 89.8 (R) enantiomer | 95 |
| 9 | 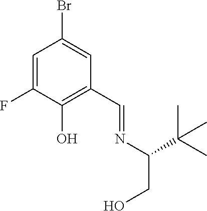 [SMILES: CC(C)(C)[C@H](CO)/N=C/c1cc(Br)cc(F)c1O] | 99.6 (R) enantiomer | 100 |
| 10 | 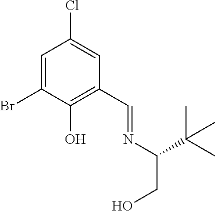 [SMILES: CC(C)(C)[C@H](CO)/N=C/c1cc(Cl)cc(Br)c1O] | 92.4 (R) enantiomer | 99 |
| 11 | 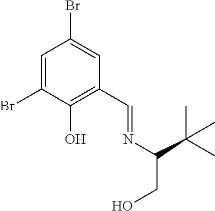 | 96.3 (S) enantiomer | 100 |

### Example 12: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

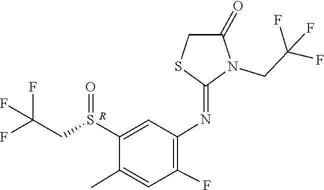

In the reaction vessel 10 ml of toluene, 19.2 mg (0.8 mmol) of lithium hydroxide and 97.7 mg (0.8 mmol) of benzoic acid were initially charged and stirred for 10 minutes at 20° C. 141.3 mg (0.4 mmol) of iron(III) acetylacetonate and 234 mg (0.8 mmol) of 2,4-dichloro-6-[(E)-{[(2R)-1-hydroxy-3,3-dimethylbutan-2-yl]imino}methyl]phenol were subsequently added. This was followed by rinsing with 2 ml of toluene. The reaction mixture was cooled to 5° C. and 32.34 g (20 mmol) of a 26.0% toluenic solution of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one were subsequently added. 5.36 g (50 mmol) of 31.8% hydrogen peroxide were then added over 30 minutes at 5° C. Reaction monitoring by HPLC after 4 hours reaction time indicated 100% conversion. The yield of title compound according to quantitative HPLC was 95.4% of theory. The ee value of the title compound was 98.1%.

### Examples 13 and 14

The synthesis described hereinabove in Example 12 was repeated with different additives. The results are reported in Table 2 which follows.

TABLE 2
Oxidation of (2Z)-2-({2-fluoro-4-methyl-5-[(2,2,2-trifluoroethyl)sulfanyl]phenyl}imino)-
3-(2,2,2-trifluoroethyl)-1,3-thiazolidin-4-one according to Example 12 in the presence os 2 mole
equivalents (based on iron (III) acetylacetonate) of different additives:

| | | | Conversion | Yield |
|---|---|---|---|---|
| Example | Additive | ee [%] | [%] | [% of th.] |
| 13 | 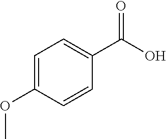 | 99.3 | 100 after 2.5 h | 95 after 4 h |
| 14 | 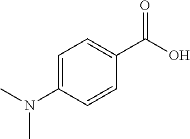 | >99.9 | 100 after 2.5 h | 94 after 4 h |

### Example 15: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

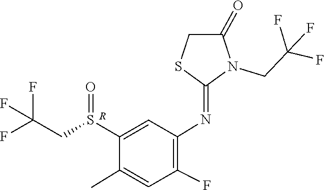

In the reaction vessel 15 ml of toluene, 24 mg (1 mmol) of lithium hydroxide and 165.2 mg (1 mmol) of 4-dimethylaminobenzoic acid were initially charged and stirred for 10 minutes at 20° C. 176.6 mg (0.5 mmol) of iron(III) acetylacetonate and 292.5 mg (1 mmol) of 2,4-dichloro-6-[(E)-{[(2R)-1-hydroxy-3,3-dimethylbutan-2-yl]imino}methyl]phenol were subsequently added. This was followed by rinsing with 2 ml of toluene. The reaction mixture was cooled to 5° C. and 40.42 g (25 mmol) of a 26.0% toluenic solution of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one were subsequently added. 6.7 g (62.5 mmol) of 31.8% hydrogen peroxide were then added over 30 minutes at 5° C. Reaction monitoring by HPLC after 2.5 hours reaction time indicated 100% conversion. The yield of title compound according to quantitative HPLC after a reaction time of 3.5 hours was 95.6% of theory. The ee value of the title compound was >99.9%.

### Examples 16 and 17

The synthesis described hereinabove in Example 15 was repeated with different molar ratios of iron(III) acetylacetonate, ligand and 4-dimethylaminobenzoic acid/LiOH based on the amount of starting compound. The results are reported in Table 3 which follows.

TABLE 3
Oxidation of (2Z)-2-({2-fluoro-4-methyl-5-[(2,2,2-
trifluoroethyl)sulfanyl]phenyl}imino)-3-(2,2,2-trifluoroethyl)-
1,3-thiazolidin-4-one according to Example 15 in the presence
of different molar ratios of iron(III) acetylacetonate,
ligand and 4-dimethylaminobenzoic acid/LiOH based on
the amount of starting compound.

| | Fe/L/4- | | | |
|---|---|---|---|---|
| | dimethylaminobenzoic | | | Yield |
| | acid/LiOH | ee | Conversion | [% of |
| Example | [mol %] | [%] | [%] | th.] |
| 16 | 2/4/8/8 | >99.9 | 100 | 94.3 |
| | | | after 3.5 h | after 3.5 h |
| 17 | 1/4/4/4 | 99.4 | 100 | 96.2 |
| | | | after 22 h | after 22 h |

L = ligand

### Example 18: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

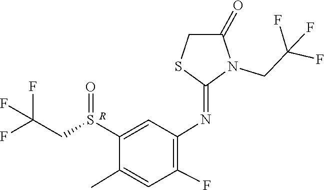

In the reaction vessel 265 mg (0.75 mmol) of iron(III) acetylacetonate, 437 mg (1.50 mmol) of 2,4-dichloro-6-[(E)-{[(2R)-1-hydroxy-3,3-dimethylbutan-2-yl]imino}methyl]phenol and 216 mg (1.50 mmol) of sodium benzoate in 9 ml of technical-grade xylene mixture were initially charged and stirred at 15° C. for 10 minutes. A solution of 7.25 g of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in 15 ml of technical-grade xylene mixture (86.9%, 15.0 mmol) were subsequently added dropwise. 4.25 g (37.5 mmol) of 30% hydrogen peroxide solution were then added at 15° C. over one hour. Reaction monitoring by HPLC after 1 hour reaction time indicated complete conversion. The reaction mixture was stirred at 15° C. for 18 h and then admixed with 7.81 g (30.0 mmol) of 40% sodium hydrogensulfite solution and stirred for 30 minutes. After addition of a further 15 ml of water the phases were separated and the aqueous phase was extracted with 5 ml of xylene. Analysis of the combined xylene phases by quantitative HPLC indicated a quantitative yield. The ee value of the title compound was >99.9%.

### Example 19: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

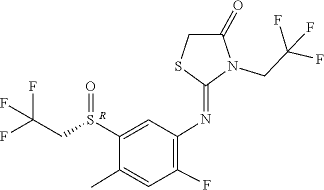

In the reaction vessel 177 mg (0.50 mmol) of iron(III) acetylacetonate, 293 mg (1.00 mmol) of 2,4-dichloro-6-[(E)-{[(2R)-1-hydroxy-3,3-dimethylbutan-2-yl]imino}methyl]phenol, 165 mg (1.00 mmol) of 4-dimethylaminobenzoic acid and 24 mg (1.00 mmol) of lithium hydroxide in 15 ml of toluene were initially charged. 40.42 g of a 26.0% solution of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (25.0 mmol) in toluene were subsequently added, followed by rinsing with a further 2 ml of toluene. 6.50 g (62.5 mmol) of 32.7% hydrogen peroxide solution were added at 5° C. over 30 minutes. The reaction mixture was stirred at 5° C. for 2 h and reaction monitoring by HPLC revealed complete conversion. 32.5 g (62.5 mmol) of 20% sodium hydrogensulfite solution were slowly added dropwise at 20° C., the emulsion was stirred overnight and the phases were subsequently separated. Analysis of the toluene phase by quantitative HPLC indicated a yield of 96.0% of theory. The ee value of the title compound was 99.6%.

### Examples 20 to 22

The synthesis described hereinabove in Example 19 was repeated with different bases. The results are reported in Table 4 which follows.

TABLE 4
Oxidation of (2Z)-2-({2-fluoro-4-methyl-5-[(2,2,2-
trifluoroethyl)sulfanyl]phenyl}imino)-3-(2,2,2-
trifluoroethyl)-1,3-thiazolidin-4-one according to
Example 19 in the presence of different bases.

| | Base | ee | Conversion | Yield |
|---|---|---|---|---|
| Example | [mol %] | [%] | [%] | [% of th.] |
| 20 | NaOH [4.0] | 99.5 | 100 | 96.4 |
| | | | after 2 h | after 2 h |
| 21 | KOH [4.0] | 99.5 | 100 | 96.7 |
| | | | after 2 h | after 2 h |
| 22 | 25% NH₃ [4.0] | >99.9 | 100 | 96.3 |
| | | | after 20 h | after 20 h |

### Example 23: Synthesis of (2Z)-2-({4-fluoro-2-methyl-5-[(R)-(2,2,2-trifluoroethyl)sulfinyl]phenyl}imino)-3-(2,2,2-trifluoroethyl)-1,3-thiazolidin-4-one

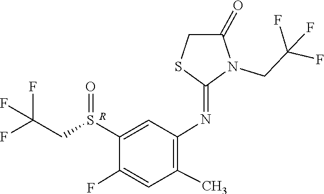

In a 2 l reactor 1000 ml of toluene, 3.335 g (11.5 mmol) of 2,4-dichloro-6-[(E)-{[(2R)-1-hydroxy-3,3-dimethylbutan-2-yl]imino}methyl]phenol, 1.656 g (11.5 mmol) of sodium benzoate and 2.03 g (5.75 mmol) of iron(III) acetylacetonate were initially charged at 15° C. and stirred for 1 hour. 80.5 g (191.5 mmol) of (2Z)-2-({4-fluoro-2-methyl-5-[(2,2,2-trifluoroethyl)sulfanyl]phenyl}imino)-3-(2,2,2-trifluoroethyl)-1,3-thiazolidin-4-one were subsequently added and 60.3 g (478.8 mmol) of 27% hydrogen peroxide were then slowly added. After a reaction time of 165 minutes the reaction mixture was admixed with 93 ml of a 40% sodium bisulfite solution and 240 ml of water and stirred at 20° C. for 30 minutes. The phases were separated and the organic phase was concentrated. The resulting residue was purified by chromatography on silica gel (cyclohexane/ethyl acetate 2:1) to afford 76.4 g of a viscous oil which according to HPLC had a purity of 97% (a/a), corresponding to a yield of 88.7% of theory. An ee value of 97.2% was determined.

1H-NMR (600 MHz, d-DMSO): δ=2.2 (s, 3H), 4.14-4.2 (m, 1H), 4.22 (s, 2H), 4.24-4.3 (m, 1H), 4.6 (m, 2H), 7.29 (d, J=6.3 Hz, 1H), 7.4 (d, J=10.3 Hz, 1H) ppm.

### Example 24: Synthesis of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one

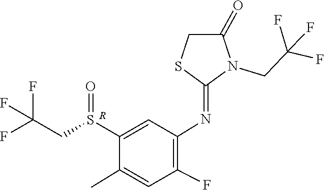

In a 1 l reaction vessel fitted with an impeller stirrer 900 g (mass fraction: 25.4%; 544 mmol) of a solution of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in toluene were initially charged. 10.45 g (21.75 mmol) of sodium benzoate, 6.428 g (21.75 mmol) of 2,4-dichloro-6-[(E)-{[(2R)-1-hydroxy-3,3-dimethylbutan-2-yl]imino}methyl]phenol and a solution of 3.841 g (10.88 mmol) of iron(III) acetylacetonate in 76 g of toluene were added. The mixture was then stirred at room temperature for 15 min before being cooled to 5° C. 105.7 g (1.087 mol) of an aqueous hydrogen peroxide solution (mass fraction: 35%) were then added over 2 h at an internal temperature of 5° C. to 9° C. The progress of the reaction was monitored by HPLC and once addition was complete the reaction mixture was stirred at 5° C. for 4 h. 174.1 g of an aqueous sodium bisulfite solution were carefully added dropwise to the reaction mixture over 30 min (mass fraction: 39%). It was ensured that the temperature of the reaction solution did not exceed 20° C. The reaction solution was subsequently heated to 20° C. and stirred for 1 h. The phases were separated and the organic phase was washed with 200 g of water at 40° C. After renewed phase separation the organic phase was analysed and the mass fraction of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one was determined as 22.2%. This corresponds to a crude yield of 95% of theory. The optical purity according to HPLC on a chiral phase was ee=99.3%. The toluene was then distilled off completely at reduced pressure and elevated temperature. The temperature was increased to 100° C. and the pressure reduced to 30 mbar. The obtained melt was then cooled to 80° C. and 102 g of 3-methyl-1-butanol were added. The mixture was then cooled to 40° C., seeded with 1.1 g of crystalline (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one and stirred at room temperature for 1 h. 306 g of methylcyclohexane were added to the obtained suspension over 1 h. The suspension was then cooled to 20° C. over 2 h and stirred at this temperature for a further hour. The suspension was filtered and the reaction vessel was rinsed out with a quantity of mother liquor. The obtained filtercake was washed with 215 g of a 3:1 mixture of methylcyclohexane and 3-methyl-1-butanol and with 215 g of pure methylcyclohexane. Both washes were performed at 20° C. as displacement washes. The filtercake was subsequently dried at 50° C. and a reduced pressure of 20 mbar. This afforded 206.8 g of (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one. The yield was 84% of theory. The ee value was determined as greater than 99.9%. It was also possible to perform the crystallization using 1-butanol instead of 3-methyl-1-butanol.

### Comparative Examples

The synthesis described in principle in Example 2 was performed under different conditions. The results are summarized in Table 5.

TABLE 5
Oxidation of (2Z)-2-({2-fluoro-4-methyl-5-[(2,2,2-trifluoroethyl)sulfanyl]phenyl}imino)-
3-(2,2,2-trifluoroethyl)-1,3-thiazolidin-4-one according to Example 2 under different conditions

| | Fe/L/additive/ | | | |
|---|---|---|---|---|
| Comparative | LiOH | | Conversion | Yield |
| example | [mol %] | ee [%] | [%] | [% of th.] |
| 1 | 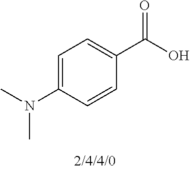 | 91.0 | — | 33 after 20 h |
| 2 | 2/4/0/4 | 87.8 | — | 27 |
| | | | | after 20 h |

L = ligand

1. A Process for preparing a 2-(phenylimino)-1,3-thiazolidin-4-one sulfoxide derivative of formula (I) in enantiomerically pure or enantiomerically enriched form, 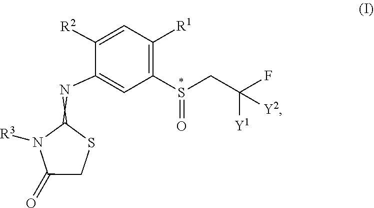
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro, and
R³ is hydrogen or optionally substituted C₆-C₁₀-aryl, (C₁-C₁₂)alkyl or (C₁-C₁₂)haloalkyl,
wherein the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, optionally from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy,
comprising reacting a sulfide of formula (II) 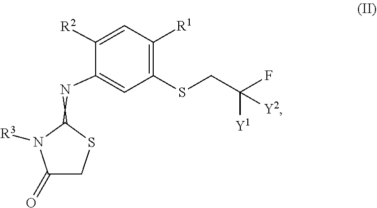
in which Y¹, Y², R¹, R² and R³ are as defined above,
in the presence of an enantiomerically enriched chiral catalyst, an additive which is the salt of an organic acid and an oxidizing agent.

2. The process according to claim 1, wherein the enantiomeric ratio is 50.5:49.5 to 100:0 (R):(S) or (S):(R) enantiomer.

3. The process according to claim 1, wherein
Y¹ and Y² are each independently fluorine, chlorine or hydrogen,
R¹ and R² are each independently fluorine, chlorine, (C₁-C₃)alkyl or hydrogen and
R³ is hydrogen or optionally substituted phenyl, (C₁-C₆)alkyl or (C₁-C₆)haloalkyl, wherein the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy.

4. The process according to claim 1, wherein
Y¹ and Y² are each independently fluorine or hydrogen,
R¹ and R² are each independently fluorine, chlorine, hydrogen or methyl and
R³ is hydrogen, (C₁-C₆)alkyl or (C₁-C₆)haloalkyl.

5. The process according to claim 1, wherein
Y¹ and Y² are fluorine,
R¹ and R² are each independently fluorine or methyl and
R³ is (C₁-C₆)haloalkyl.

6. The process according to claim 1, wherein
Y¹ and Y² are fluorine, R¹ is methyl, R² is fluorine and R³ is CH₂CF₃.

7. The process according to claim 1, wherein the oxidizing agent employed is selected from organic or inorganic peroxides.

8. The process according to claim 1, wherein the chiral catalyst employed is a chiral metal-ligand complex, wherein the metal is a transition metal or transition metal derivative.

9. The process according to claim 8, wherein the ligand is a compound of formula (III) 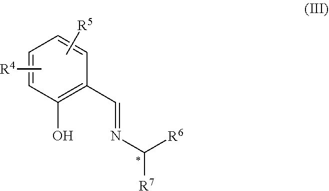
in which
R⁴ and R⁵ are each independently hydrogen, (C₁-C₆)alkyl, (C₁-C₆)haloalkyl, (C₁-C₆)alkylphenyl, phenyl, halogen, cyano, nitro, cyano(C₁-C₆)alkyl, hydroxy(C₁-C₆)alkyl, (C₁-C₆)alkoxycarbonyl(C₁-C₆)alkyl, (C₁-C₆)alkoxy, (C₁-C₆)haloalkoxy or (C₁-C₆)alkoxy(C₁-C₆)alkyl,
R⁶ is (C₁-C₆)alkyl, halogen-, cyano-, nitro-, amino-, hydroxy- or phenyl-substituted (C₁-C₆)alkyl, carboxyl, carbonyl(C₁-C₆)alkyl, (C₁-C₆)alkoxycarbonyl(C₁-C₆)alkyl, (C₁-C₆)alkoxy(C₁-C₆)alkyl, (C₁-C₆)alkoxy or di(C₁-C₆)alkoxy(C₁-C₆)alkyl,
R⁷ is hydrogen, (C₁-C₆)alkyl, (C₁-C₆)alkylphenyl, aryl or aryl(C₁-C₆)alkyl, and chiral carbon atoms are identified by *.

10. The process according to claim 8, wherein the ligand is a compound of formula (IIIa) 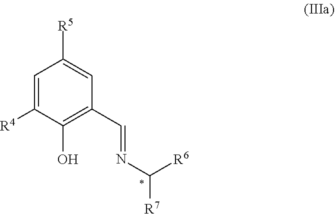
in which
R⁴ and R⁵ are each independently hydrogen, (C₁-C₆)alkyl, (C₁-C₆)alkylphenyl, phenyl, halogen, cyano, nitro, cyano(C₁-C₆)alkyl, hydroxy(C₁-C₆)alkyl, (C₁-C₆)alkoxycarbonyl(C₁-C₆)alkyl or (C₁-C₆)alkoxy(C₁-C₆)alkyl,
R⁶ is (C₁-C₆)alkyl, halogen-, cyano-, nitro-, amino-, hydroxy- or phenyl-substituted (C₁-C₆)alkyl, carboxyl, carbonyl(C₁-C₆)alkyl, (C₁-C₆)alkoxycarbonyl(C₁-C₆)alkyl, (C₁-C₆)alkoxy(C₁-C₆)alkyl, (C₁-C₆)alkoxy or di(C₁-C₆)alkoxy(C₁-C₆)alkyl,
R⁷ is hydrogen, (C₁-C₆)alkyl, (C₁-C₆)alkylphenyl, aryl or aryl(C₁-C₆)alkyl, and chiral carbon atoms are identified by *.

11. The process according to claim 9, wherein R⁴ and R⁵ are each independently hydrogen or chlorine, R⁶ represents hydroxy-substituted C₁-alkyl and R⁷ is tert-butyl.

12. The process according to claim 8, wherein the transition metal is molybdenum, zirconium, iron, manganese or titanium or a derivative thereof.

13. The process according to claim 8, wherein the transition metal is iron or an iron derivative.

14. The process according to claim 8, wherein the transition metal is titanium or a titanium derivative.

15. The process according to claim 8, wherein the transition metal derivative is a titanium or iron halide, a titanium or iron carboxylate or a titanium or iron acetylacetonate.

16. The process according to claim 8, wherein the chiral metal-ligand complex is employed in an amount of 0.01 to 20 mol % based on the sulfide of formula (II).

17. The process according to claim 1, wherein the additive is an alkali metal salt of the organic acid, optionally the lithium, sodium or potassium salt thereof.

18. The process according to claim 1, wherein the additive is one of formula (IV) 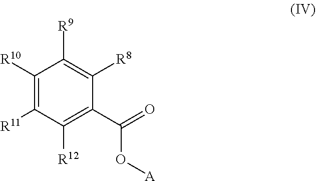
wherein in formula (IV)
R⁸, R⁹, R¹⁰, R¹¹ and R¹² are each independently hydrogen, (C₁-C₆)alkyl, (C₁-C₆)haloalkyl, (C₁-C₆)haloalkoxy, (C₁-C₆)alkylphenyl, phenyl, halogen, cyano, nitro, (C₁-C₆)alkoxy, cyano(C₁-C₆)alkyl, hydroxy(C₁-C₆)alkyl, (C₁-C₆)alkoxycarbonyl(C₁-C₆)alkyl, (C₁-C₆)alkoxy(C₁-C₆)alkyl or aminodi(C₁-C₆)alkyl and
A is lithium, sodium, potassium or an NR¹³R¹⁴R¹⁵R¹⁶ radical, wherein
R¹³, R¹⁴, R¹⁵ and R¹⁶ are each independently hydrogen, benzyl or (C₁-C₆)alkyl.

19. The process according to claim 18, wherein
R⁸, R⁹, R¹¹ and R¹² are hydrogen,
R¹⁰ is hydrogen or methoxy or dimethylamino and
A is lithium, sodium or potassium.

20. The process according to claim 1, wherein the additive is employed in an amount of 0.1 to 20 mol % based on the sulfide of formula (II).

21. The process according to claim 1, wherein said process is performed in the presence of a solvent selected from the group consisting of methylene chloride, chloroform, 1,2-dichloroethane, chlorobenzene, 1,2-dichlorobenzene, acetonitrile, acetone, toluene, anisole, o-xylene, m-xylene, p-xylene, ethylbenzene, ethyl acetate, methyl tert-butyl ether (MTBE), tetrahydrofuran (THF), N,N-dimethylacetamide (DMAc), N,N-dimethylformamide (DMF), ethanol and mixtures thereof.

22. The process according to claim 1, further comprising crystallization of the compound of formula (I) from organic solvent or a mixture of organic solvent with water is carried out.

23. The process according to claim 1, wherein the molar ratio of oxidizing agent to the sulfide of formula (II) is in the range from 0.9:1 to 5:1.

24. The process according to claim 1, wherein the oxidizing agent is hydrogen peroxide.

25. An Enantiomerically pure or enantiomerically enriched 2-(phenylimino)-1,3-thiazolidin-4-one sulfoxide derivatives of formula (I) as defined in claim 1, wherein the enantiomeric ratio is 50.5:49.5 to 100:0 (R):(S) enantiomer.

26. An Enantiomerically pure or enantiomerically enriched 2-(phenylimino)-1,3-thiazolidin-4-one sulfoxide derivatives of formula (I) as defined in claim 1, wherein the enantiomeric ratio is 50.5:49.5 to 100:0 (S):(R) enantiomer.
